SEQUENCE LISTING (18 sequences)

SEQ ID NO: 1 - protein, 10 aa, Artificial Sequence
  synthetic construct
CGLVPAGSGP

SEQ ID NO: 2 - protein, 17 aa, Artificial Sequence
  synthetic construct
CGLVPAGSGPSAGSSAC

SEQ ID NO: 3 - protein, 882 aa, Artificial Sequence
  synthetic construct
MPFELPALPYPYDALEPHIDKETMNIHHTKHHNTYVTNLNAALEGHPDLQNKSLEELLSNLEALPESIRTAVRNNGGGHANHSLFWTILSPNGGGEPTGELADAINKKFGSFTAFKDEFSKAAAGRFGSGWAWLVVNNGELEITSTPNQDSPIMEGKTPILGLDVWEHAYYLKYQNRRPEYIAAFWNVVNWDEVAKRYSEAKPKSGSCGLVPRGSGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 4 - protein, 862 aa, Artificial Sequence
  synthetic construct
MPFELPALPYPYDALEPHIDKETMNIHHTKHHNTYVTNLNAALEGHPDLQNKSLEELLSNLEALPESIRTAVRNNGGGHANHSLFWTILSPNGGGEPTGELADAINKKFGSFTAFKDEFSKAAAGRFGSGWAWLVVNNGELEITSTPNQDSPIMEGKTPILGLDVWEHAYYLKYQNRRPEYIAAFWNVVNWDEVAKRYSEAKPKSGSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 5 - protein, 907 aa, Artificial Sequence
  synthetic construct
MLSRAVCGTSRQLAPALGYLGSRQKHSRGSPALPYPYDALEPHIDKETMNIHHTKHHNTYVTNLNAALEGHPDLQNKSLEELLSNLEALPESIRTAVRNNGGGHANHSLFWTILSPNGGGEPTGELADAINKKFGSFTAFKDEFSKAAAGRFGSGWAWLVVNNGELEITSTPNQDSPIMEGKTPILGLDVWEHAYYLKYQNRRPEYIAAFWNVVNWDEVAKRYSEAKPKSGSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 6 - protein, 887 aa, Artificial Sequence
  synthetic construct
MLSRAVCGTSRQLAPALGYLGSRQKHSRGSPALPYPYDALEPHIDKETMNIHHTKHHNTYVTNLNAALEGHPDLQNKSLEELLSNLEALPESIRTAVRNNGGGHANHSLFWTILSPNGGGEPTGELADAINKKFGSFTAFKDEFSKAAAGRFGSGWAWLVVNNGELEITSTPNQDSPIMEGKTPILGLDVWEHAYYLKYQNRRPEYIAAFWNVVNWDEVAKRYSEAKPKSGSCGLVPRGSGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 685 aa, Artificial Sequence
  synthetic construct
MPFELPALPYPYDALEPHIDKETMNIHHTKHHNTYVTNLNAALEGHPDLQNKSLEELLSNLEALPESIRTAVRNNGGGHANHSLFWTILSPNGGGEPTGELADAINKKFGSFTAFKDEFSKAAAGRFGSGWAWLVVNNGELEITSTPNQDSPIMEGKTPILGLDVWEHAYYLKYQNRRPEYIAAFWNVVNWDEVAKRYSEAKPKSGSCGIEGRAPGPGSSVGSSLSCINGLFGAIAGFIENGWEGMIDGWYGTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 8 - protein, 645 aa, Artificial Sequence
  synthetic construct
GSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 9 - protein, 665 aa, Artificial Sequence
  synthetic construct
GSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 10 - protein, 448 aa, Artificial Sequence
  synthetic construct
GSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSVYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 11 - protein, 605 aa, Artificial Sequence
  synthetic construct
GSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSVYNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 12 - protein, 660 aa, Artificial Sequence
  synthetic construct
GSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 13 - protein, 210 aa, Corynebacterium diphtheriae
SVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRS

SEQ ID NO: 14 - protein, 1092 aa, Clostridium botulinum
LVSKFENSVKNSNKNYFTINGLMGYYFENDFFNLNIISPTLDGNLTFSKEDINSILGNKIIKSARWIGLIKPSITGEYILSTNSPNCRVELNGEIFNLSLNTSNTVNLIQGNVYDIRIEQLMSENQLLKNYEGIKLYWETSDIIKEIIPSEVLLKPNYSNTNEKSKFIPNNTLFSNAKLKANANRDTDRDGIPDEWEINGYTVMNQKAVAWDDKFAANGYKKYVSNPFKPCTANDPYTDFEKVSGQIDPSVSMVARDPMISAYPIVGVQMERLVVSKSETITGDSTKSMSKSTSHSSTNINTVGAEVSGSLQLAGGIFPVFSMSASANYSHTWQNTSTVDDTTGESFSQGLSINTGESAYINPNIRYYNTGTAPVYNVTPTTTIVIDKQSVATIKGQESLIGDYLNPGGTYPIIGEPPMALNTMDQFSSRLIPINYNQLKSIDNGGTVMLSTSQFTGNFAKYNSNGNLVTDGNNWGPYLGTIKSTTASLTLSFSGQTTQVAVVAPNFSDPEDKTPKLTLEQALVKAFALEKKNGKFYFHGLEISKNEKIQVFLDSNTNNDFENQLKNTADKDIMHCIIKRNMNILVKVITFKENISSINIINDTNFGVQSMTGLSNRSKGQDGIYRAATTAFSFKSKELKYPEGRYRMRFVIQSYEPFTTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 15 - protein, 1032 aa, Clostridium botulinum
LVSKFENSVKNSNKNYFTINGLMGYYFENDFFNLNIISPTLDGNLTFSKEDINSILGNKIIKSARWIGLIKPSITGEYILSTNSPNCRVELNGEIFNLSLNTSNTVNLIQGNVYDIRIEQLMSENQLLKNYEGIKLYWETSDIIKEIIPSEVLLKPNYSNTNEKSKFIPNNTLFSNAKLKANANRDTDRDGIPDEWEINGYTVMNQKAVAWDDKFAANGYKKYVSNPFKPCTANDPYTDFEKVSGQIDPSVSMVARDPMISAYPIVGVQMERLVVSKSETITGDSTKSMSKSTSHSSTNINTVGAEVSGSLQLAGGIFPVFSMSASANYSHTWQNTSTVDDTTGESFSQGLSINTGESAYINPNIRYYNTGTAPVYNVTPTTTIVIDKQSVATIKGQESLIGDYLNPGGTYPIIGEPPMALNTMDQFSSRLIPINYNQLKSIDNGGTVMLSTSQFTGNFAKYNSNGNLVTDGNNWGPYLGTIKSTTASLTLSFSGQTTQVAVVAPNFSDPEDKTPKLTLEQALVKAFALEKKNGKFYFHGLEISKNEKIQVFLDSNTNNDFENQLKNTADKDIMHCIIKRNMNILVKVITFKENISSINTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 16 - protein, 1112 aa, Artificial Sequence
  synthetic construct
LVSKFENSVKNSNKNYFTINGLMGYYFENDFFNLNIISPTLDGNLTFSKEDINSILGNKIIKSARWIGLIKPSITGEYILSTNSPNCRVELNGEIFNLSLNTSNTVNLIQGNVYDIRIEQLMSENQLLKNYEGIKLYWETSDIIKEIIPSEVLLKPNYSNTNEKSKFIPNNTLFSNAKLKANANRDTDRDGIPDEWEINGYTVMNQKAVAWDDKFAANGYKKYVSNPFKPCTANDPYTDFEKVSGQIDPSVSMVARDPMISAYPIVGVQMERLVVSKSETITGDSTKSMSKSTSHSSTNINTVGAEVSGSLQLAGGIFPVFSMSASANYSHTWQNTSTVDDTTGESFSQGLSINTGESAYINPNIRYYNTGTAPVYNVTPTTTIVIDKQSVATIKGQESLIGDYLNPGGTYPIIGEPPMALNTMDQFSSRLIPINYNQLKSIDNGGTVMLSTSQFTGNFAKYNSNGNLVTDGNNWGPYLGTIKSTTASLTLSFSGQTTQVAVVAPNFSDPEDKTPKLTLEQALVKAFALEKKNGKFYFHGLEISKNEKIQVFLDSNTNNDFENQLKNTADKDIMHCIIKRNMNILVKVITFKENISSINIINDTNFGVQSMTGLSNRSKGQDGIYRAATTAFSFKSKELKYPEGRYRMRFVIQSYEPFTKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 17 - protein, 1052 aa, Artificial Sequence
  synthetic construct
LVSKFENSVKNSNKNYFTINGLMGYYFENDFFNLNIISPTLDGNLTFSKEDINSILGNKIIKSARWIGLIKPSITGEYILSTNSPNCRVELNGEIFNLSLNTSNTVNLIQGNVYDIRIEQLMSENQLLKNYEGIKLYWETSDIIKEIIPSEVLLKPNYSNTNEKSKFIPNNTLFSNAKLKANANRDTDRDGIPDEWEINGYTVMNQKAVAWDDKFAANGYKKYVSNPFKPCTANDPYTDFEKVSGQIDPSVSMVARDPMISAYPIVGVQMERLVVSKSETITGDSTKSMSKSTSHSSTNINTVGAEVSGSLQLAGGIFPVFSMSASANYSHTWQNTSTVDDTTGESFSQGLSINTGESAYINPNIRYYNTGTAPVYNVTPTTTIVIDKQSVATIKGQESLIGDYLNPGGTYPIIGEPPMALNTMDQFSSRLIPINYNQLKSIDNGGTVMLSTSQFTGNFAKYNSNGNLVTDGNNWGPYLGTIKSTTASLTLSFSGQTTQVAVVAPNFSDPEDKTPKLTLEQALVKAFALEKKNGKFYFHGLEISKNEKIQVFLDSNTNNDFENQLKNTADKDIMHCIIKRNMNILVKVITFKENISSINKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 18 - protein, 10 aa, Artificial Sequence
  synthetic construct
CGIEGRAPGP

(19) United States
(12) Patent Application Publication
(10) Pub. No.: US 2012/0128700 A1
(43) Pub. Date: May 24, 2012

(54) CONSTRUCTS FOR DELIVERY OF THERAPEUTIC AGENTS TO NEURONAL CELLS

(75) Inventors: Clifford Charles SHONE, Abingdon (GB); John Mark SUTTON, Abingdon (GB); Nigel SILMAN, Abingdon (GB)

(73) Assignee: SYNTAXIN LIMITED, Abingdon (GB)

(21) Appl. No.: 13/363,544

(22) Filed: Feb. 1, 2012

Related U.S. Application Data
(63) Continuation of application No. 11/798,909, filed on May 17, 2007.
Application No. 11/798,909 is a continuation of application No. 10/130,973, filed on Jun. 25, 2002, now Pat. No. 7,368,532.

(30) Foreign Application Priority Data
Dec. 2, 1999 (GB) 9928530.6
Apr. 7, 2000 (GB) 0008658.7

(51) Int. Cl. A61K 39/395; A61P 25/00; C12N 5/079; C07K 17/02; C07K 19/00

(52) U.S. Cl. 424/178.1; 530/391.1; 530/387.3; 435/375

(57) ABSTRACT

A non-toxic polypeptide, for delivery of a therapeutic agent to a neuronal cell, comprises a binding domain that binds to the neuronal cell, and a translocation domain that translocates the therapeutic agent into the neuronal cell, wherein the translocation domain is not a H_{N} domain of a clostridial toxin and is not a fragment or derivative of a H_{N} domain of a clostridial toxin.

This application is a continuation of U.S. patent application Ser. No. 11/798,909, filed on May 17, 2007, pending, which is a continuation of U.S. patent application Ser. No. 10/130,973, now U.S. Pat. No. 7,368,532, which is a national phase entry of PCT/GB2000/04644, filed on Dec. 4, 2000. Each of the above applications is incorporated by reference herein in its entirety.

Pursuant to the provisions of 37 C.F.R. §1.52(e)(5), the sequence listing text file named 82046_Sequence_Listing.txt, created on Nov. 2, 2011 and having a size of 102,786 bytes, and which is being submitted herewith, is incorporated by reference herein in its entirety.

The present invention relates to constructs for delivering therapeutic substances to neuronal cells, to manufacture and use thereof, and in particular to constructs based on clostridial neurotoxins.

There are presently few effective treatments for major disorders of the central nervous system. Such disorders include neurodegenerative diseases, stroke, epilepsy, brain tumours, infections and HIV encephalopathy, and sufferers of these diseases far outnumber the morbidity of cancer and heart disease. The number of sufferers for CNS disorders such as stroke and the neurodegenerative diseases is set to grow, particularly in developed countries where the average age of the population is increasing. As our understanding of brain pharmacology increases and the underlying pathologies of diseases are elucidated, potential therapeutic strategies become apparent. All these treatments, however, face the formidable problem of efficient delivery of therapeutics to the various neuronal cell populations involved. Vectors which can effect efficient delivery to neuronal cells are thus required for a broad range of therapeutic substances, including drugs, enzymes, growth factors, therapeutic peptides and genes.

Ischemia/reperfusion injury induced by stroke or injury is on notable example in which rapid and efficient delivery of therapeutic agents would afford considerable benefit. Neurons injured by trauma or ischemia produce elevated levels of free oxygen radicals and release large amount of glutamate. These substances in high concentration are toxic to both neurons and surrounding cells which potentiate and amplify the damage process. Agents such as superoxide dismutase or glutamine synthetase which reduce the levels of these toxic substances have been shown to reduce the neuronal cell death in a variety of in vitro and in vivo ischemia models (Gorovits et al. PNAS (1997) 94, 7024-7029; Francis et al. Experimental Neurology (1997) 146, 435-443; Lim et al. Ann. Thorac. Surg. (1986) 42, 282-286; Cuevas et al. Acta Anat. (1990) 137, 303-310). A major problem in the use of such therapies is in the delivery of useful concentrations of the active agent to the site of trauma. Specific neuronal vectors could therefore play an important role in targeting such compounds to neuronal cells.

Peripheral nervous system disorders, such as motor neuron disease, are further examples of diseases which would benefit from the targeted delivery of therapeutic agents. Such therapies could take the form of drug delivery or DNA delivery via gene therapy strategies.

Gene therapy holds considerable promise for the treatment of neurodegenerative diseases such as Parkinson's and Alzheimer's diseases. Most of the currently available viral and non-viral gene delivery vectors lack tissue specificity which reduces both their efficiency and safety of use. Suitable neuronal cell-specific targeting ligands are therefore required for a broad range of gene vectors to enable effective treatments for neuronal diseases to be developed.

The botulinum neurotoxins are a family of protein toxins whose primary site of action is the neuromuscular junction where they block the release of the transmitter acetylcholine. The action of these toxins on the peripheral nervous system of man and animals results in the syndrome botulism, which is characterised by widespread flaccid muscular paralysis (Shone (1986) in ‘Natural Toxicants in Foods’, Editor D. Watson, Ellis Harwood, UK). Each of the botulinum neurotoxins consists of two disulphide-linked subunits; a 100 kDa heavy subunit which plays a role in the initial binding and internalisation of the neurotoxin into the nerve ending (Dolly et. al. (1984) Nature, 307, 457-460) and a 50 kDa light subunit which acts intracellularly to block the exocytosis process (McInnes and Dolly (1990) Febs Lett., 261, 323-326; de Paiva and Dolly (1990) Febs Lett., 277, 171-174).

The clostridial neurotoxins are potent inhibitors of calcium-dependent neurotransmitter secretion in neuronal cells. They are currently considered to mediate this activity through a specific endoproteolytic cleavage of at least one of three vesicle or pre-synaptic membrane associated proteins VAMP, syntaxin or SNAP-25 which are central to the vesicle docking and membrane fusion events of neurotransmitter secretion. The neuronal cell targeting of tetanus and botulinum neurotoxins is considered to be a receptor mediated event following which the toxins become internalised and subsequently traffic to the appropriate intracellular compartment where they effect their endopeptidase activity.

Clostridial neurotoxins share a common architecture of a catalytic L-chain (LC, ca 50 kDa) disulphide linked to a receptor binding and translocating H-chain (HC, ca 100 kDa). The HC polypeptide is considered to comprise all or part of two distinct functional domains. The carboxy-terminal half of the HC, termed the H_{C} domain (ca 50 kDa), is involved in the high affinity, neurospecific binding of the neurotoxin to cell surface receptors on the target neuron, whilst the amino-terminal half, termed the H_{N} domain (ca 50 kDa), is considered to mediate the translocation of at least some portion of the neurotoxin across cellular membranes such that the functional activity of the LC is expressed within the target cell. The H_{N} domain also has the property, under conditions of low pH, of forming ion-permeable channels in lipid membranes, and this may in some manner relate to its translocation function. For botulinum neurotoxin type A (BoNT/A) these domains are considered to reside within amino acid residues 872-1296 for the H_{C}, amino acid residues 449-871 for the H_{N} and residues 1-448 for the LC.

It is therefore possible to provide functional definitions of the domains within the neurotoxin molecule, as follows:—
(A) clostridial neurotoxin light chain:—
   a metalloprotease exhibiting high substrate specificity for vesicle and/or plasma membrane associated proteins involved in the exocytotic process. In particular, it cleaves one or more of SNAP-25, VAMP (synaptobrevin/cellubrevin) and syntaxin.
(B) clostridial neurotoxin heavy chain H_{N} domain:—
   a portion of the heavy chain which enables translocation of that portion of the neurotoxin molecule such that a functional expression of light chain activity occurs within a target cell.
   the domain responsible for translocation of the endopeptidase activity, following binding of neurotoxin to its specific cell surface receptor via the binding domain, into the target cell.
   the domain responsible for formation of ion-permeable pores in lipid membranes under conditions of low pH.
(c) clostridial neurotoxin heavy chain H_{C} domain:—
   a portion of the heavy chain which is responsible for binding of the native holotoxin to cell surface receptor(s) involved in the intoxicating action of clostridial toxin prior to internalisation of the toxin into the cell.

The identity of the cellular recognition markers for these toxins is currently not understood and no specific receptor species have yet been identified although Kozaki et al. have reported that synaptotagmin may be the receptor for botulinum neurotoxin type B. It is probable that each of the neurotoxins has a different receptor.

Tetanus toxin is structurally very similar to botulinum neurotoxins but its primary site of action is the central nervous system where it blocks the release of inhibitory neurotransmitters from central synapses (Renshaw cells).

Tetanus and the botulinum neurotoxins from most of the seven serotypes, together with their derived heavy chains, have been shown to bind a wide variety of neuronal cell types with high affinities in the nM range, e.g. botulinum type B neurotoxin (Evans et al. (1986) Eur. J. Biochem. 154, 409-416).

However, a major obstacle to the use of the native clostridial heavy chain fragments as delivery vectors is that their highly aggregated state in solution prevent their adequate diffusion into body tissue and hence reduces their efficiency as targeting vectors. A further significant problem with any proposed clinical use of native tetanus toxin fragments as neuronal targeting ligands for therapeutics is the existence of circulating antibodies to the toxin in the majority of the population who have been immunized against tetanus. The presence of these antibodies is likely to reduce the efficacy of constructs based on tetanus toxin fragments. Thus, clostridial neurotoxin fragments do not offer solutions to the problems identified.

The present invention is based upon the discovery of the practical difficulties in using clostridial neurotoxin-based therapeutic compositions, and the devising of modified polypeptides and hybrid polypeptides based on clostridial neurotoxin fragments that avoid the aforementioned drawbacks.

Accordingly, a first aspect of the invention provides a non-toxic polypeptide, for delivery of a therapeutic agent to a neuronal cell, comprising:—
a binding domain that binds to the neuronal cell, and
a translocation domain that translocates the therapeutic agent into the neuronal cell,
   wherein the translocation domain is not a H_{N} domain of a clostridial neurotoxin and is not a fragment or derivative of a H_{N} domain of a clostridial toxin.

The binding domain is suitably comprised of or derived from clostridial heavy chain fragments or modified clostridial heavy chain fragments. As used herein, the term “modified clostridial heavy chain fragment” means a polypeptide fragment which retains similar biological functions to the corresponding heavy chain of a botulinum or tetanus neurotoxin but differs in its amino acid sequence and other properties compared to the corresponding heavy chain. The invention more specifically provides such constructs which are based on fragments derived from botulinum and tetanus neurotoxins.

In a further aspect, the invention also provides a polypeptide, for delivery of a therapeutic agent to a neuronal cell, comprising:—
a binding domain that binds to the neuronal cell, and
a translocation domain that translocates the therapeutic agent into the neuronal cell,
   wherein the resulting polypeptide construct is non-aggregating.

Whether the construct is an aggregating one is usually apparent from a lack of solubility of the construct, and this may be seen upon simple visual inspection of the construct in aqueous media: non-aggregating domains result in constructs of the invention that are partially or preferably totally soluble whereas aggregating domains result in non-soluble aggregates of polypeptides having apparent sizes of many tens or even hundreds the size of a single polypeptide. Generally, the construct should be non-aggregating as measured by size on gel electrophoresis, and the size or apparent size of the construct measured should preferably be less than 5.0×10⁵ daltons, more preferably less than 1.5×10⁵ daltons, with the measuring being suitably carried out on native PAGE using physiological conditions.

A still further aspect of the invention provides a polypeptide, for delivery of a therapeutic agent to a neuronal cell, comprising:—
a binding domain that binds to the neuronal cell, and
a translocation domain that translocates the therapeutic agent into the neuronal cell,
   wherein the translocation domain is selected from (1) a H_{N} domain of a diphtheria toxin, (2) a fragment or derivative of (1) that substantially retains the translocating activity of the H_{N} domain of a diphtheria toxin, (3) a fusogenic peptide, (4) a membrane disrupting peptide, (5) a H_{N} from botulinum toxin C₂ and (6) translocating fragments and derivatives of (3), (4) and (5).

It is to be noted that botulinum toxin C₂ is not a neurotoxin as it has no neuronal specificity, instead it is an enterotoxin and suitable for use in the invention to provide a non-aggregating translocation domain.

A yet further aspect of the invention provides a polypeptide, for delivery of a therapeutic agent to a neuronal cell, comprising:—
a binding domain that binds to the neuronal cell, and
a translocation domain that translocates the therapeutic agent into the neuronal cell,
   wherein the polypeptide has reduced affinity to neutralising antibodies to tetanus toxin compared with the affinity to such antibodies of native tetanus toxin heavy chain.

The above aspects may singly or in any combination be exhibited by polypeptides of the invention and thus a typical preferred polypeptide of the invention (i) lacks the neurotoxic activities of botulinum and tetanus toxins, (ii) displays high affinity to neuronal cells corresponding to the affinity of a clostridial neurotoxin for those cells, (iii) contains a domain which can effect translocation across cell membranes, and (iv) occurs in a less aggregated state than the corresponding heavy chain from botulinum or tetanus toxin in physiological buffers.

A significant advantage of the polypeptides of the invention is their non-aggregated state, thus rendering them usable as soluble polypeptides where the prior art constructs were not and overcoming most if not all of the drawbacks of previous constructs based upon clostridial neurotoxins.

The polypeptides according to the invention generally include sequences from the H_{C} domains of the botulinum and tetanus neurotoxins and these are combined with functional domains from other proteins, such that the essential functions of the native heavy chain, binding to neuronal cells, is retained. Thus, for example, the H_{C} domain of botulinum type F neurotoxin is fused to the translocation domain derived from diphtheria toxin to give a modified clostridial heavy chain fragment. Surprisingly, such polypeptides are more useful as constructs for delivering substances to neuronal cells than are the native clostridial heavy chains.

Thus, according to a preferred aspect of the invention there is provided a polypeptide having an amino acid sequence comprising (a) a sub-sequence based on the H_{C} fragment of botulinum or tetanus neurotoxin, and (b) a sub-sequence based on a translocation domain, e.g. from diphtheria toxin, that is not derived from a clostridial neurotoxin, and wherein the said polypeptide (i) lacks the neurotoxin activities of botulinum and tetanus toxins, (ii) displays high affinity to neuronal cells, (iii) contains a domain which can effect translocation across cell membranes and (iv) occurs in a less aggregated state than the corresponding heavy chain of botulinum or tetanus toxin in physiological buffers.

The modified clostridial heavy chain is suitably produced by combining the binding domain (H_{C} domain) of a clostridial neurotoxin with a non-clostridial translocation domain. Thus, for example, a modified clostridial heavy chain fragment may be constructed from the translocation domain of diphtheria toxin (residues 194-386) fused to the H_{C} domain of a botulinum toxin (e.g. type F H_{C} fragment, residues 865-1278; type A H_{C} fragment, residues 872-1296).

In another embodiment of the invention, the modified clostridial heavy chain is produced by combining the H_{C} domain of a clostridial neurotoxin with a membrane disrupting peptide which functions as a translocation domain, suitably a viral peptide. Thus, for example, a modified clostridial heavy chain fragment may be constructed by combining the H_{C} domain of a botulinum toxin with a peptide based on influenza virus haemagglutinin HA2 (residues 1-23).

The polypeptides of the invention have properties which make them useful as neuronal targeting ligands; they are non-toxic and yet retain the specific, high affinity binding to neuronal cells displayed by the botulinum or tetanus toxins. Unlike the native clostridial heavy chains, however, the modified clostridial heavy chains occur in a less aggregated state in solution which improves their access to neuronal cells. The preferred constructs are soluble in aqueous solution, in contrast to the highly aggregated state of the prior art constructs.

In another aspect of the invention, there is provided a modified tetanus heavy chain fragment which, in addition to the properties of modified heavy chains defined above, has the added advantage in that it has reduced affinity to neutralizing antibodies, present as a result of anti-tetanus inoculation, compared to the native tetanus toxin heavy chain. The polypeptides according to this aspect of the invention generally include subsequences derived from the heavy chain of tetanus toxin (residues 458-1315) and from which epitopes responsible for the immunogenicity of tetanus toxin have optionally been reduced or removed. Thus, for example, it is desirable to eliminate immunogenic epitopes associated with H_{C} domain as well as that of the H_{N} domain. Although it is possible to eliminate epitopes by deleting small numbers of amino acids (e.g. less than 20 or preferably less than 10 amino acids), it has been found that epitopes associated with immunogenicity of tetanus toxin heavy chain can be reduced more rigorously by replacing a large number of amino acid residues (e.g. at least 100, at least 200 and preferably 400 or more residues) with amino acid sequences from other toxins.

Thus according to a preferred aspect of the invention related to modified tetanus heavy chains, there is provided a polypeptide having an amino sequence comprising (a) an H_{N} domain derived from a non-clostridial source (e.g. diphtheria toxin), (b) one or more subsequences derived from the sequence of a botulinum H_{C}, and (c) one or more subsequences derived from the sequence of tetanus toxin H_{C}, and wherein said polypeptide (i) lacks the neurotoxin activities of botulinum and tetanus toxins, (ii) displays high affinity to neuronal cells corresponding to the neuronal binding of tetanus neurotoxin, (iii) contains a domain which can effect translocation across cell membranes and (iv) has low affinity to neutralizing antibodies to tetanus toxin which are present as result of anti-tetanus inoculation.

This latter modified tetanus heavy chain fragment can be produced by combining the binding domain (H_{C} domain) of tetanus neurotoxin with a non-clostridial translocation domain. Thus, for example, a modified tetanus heavy chain fragment may be constructed from the translocation domain of diphtheria toxin (residues 194-386) fused to the H_{C} domain of a tetanus toxin (residues 865-1315).

In another embodiment of the invention the modified tetanus heavy chain is derived a non-clostridial translocation domain fused to the H_{C} domain of a botulinum toxin into which the minimal domains of tetanus toxin are inserted to confer tetanus toxin-like binding activity onto the resulting hybrid. Thus, for example a modified tetanus heavy chain may be constructed from the translocation domain of diphtheria toxin (residues 194-386) fused to the H_{C} domain of a botulinum type F fragment (residues 865-1278) in which residues 1097-1273 of the latter have been replaced by homologous sequences from tetanus toxin.

The modified tetanus heavy chains have properties which make them useful as neuronal targeting ligands; they are non-toxic and yet retain the specific, high affinity binding to neuronal cells displayed by tetanus toxin. Unlike native tetanus toxin binding fragments, however, the modified clostridial binding fragments have different immunogenic properties which makes them more useful clinically. Specifically, the different immunogenic properties of the modified clostridial binding fragments of the invention significantly reduce the problems caused by existing antibodies to native tetanus toxin sequences.

While the use of modified heavy chains based on botulinum neurotoxins as neuronal targeting ligands does not suffer from the problem of pre-existing circulating antibodies, tetanus toxin is unique amongst the clostridial toxins in that it has selectivity to inhibitory neurons (e.g. Renshaw cells) and as such the modified tetanus toxin heavy chains are valuable targeting ligands for this class of neuron. Tetanus toxin also has the property that it can retrograde transport from the peripheral to the central nervous system.

In another embodiment of the invention, the modified clostridial heavy chain fragment is fused to a linker peptide via the N-terminus of the translocation domain to which a polypeptide payload may be attached. An examples of such a linker peptide is the sequence CGLVPAGSGP (SEQ ID NO:1) which contains the thrombin protease cleavage site and a cysteine residue for disulphide bridge formation. Such a peptide linker allows production of a recombinant fusion protein comprising a polypeptide therapeutic molecule fused by the linker peptide to the N-terminus of the modified clostridial heavy chain fragment. The latter single chain fusion protein may then be treated with thrombin to give a dichain protein in which the polypeptide therapeutic is linked to the translocation domain of the modified clostridial heavy chain fragment by a disulphide link. In another example of a linker peptide in which the translocation domain does not contain a free cysteine residue near its C-terminus, such as is the case when the translocation domain is a fusogenic peptide, the linker peptide contains both cysteine residues required for the disulphide bridge. An example of the latter linker peptide is the amino acid sequence: CGLVPAGSGPSAGSSAC (SEQ ID NO:2).

In another embodiment of the invention, the modified clostridial heavy chain is linked to a polypeptide which may be an enzyme, growth factor, protein or peptide which has therapeutic benefits when delivered to neuronal cells. The polypeptide may be linked to the modified clostridial heavy chain by chemical means. Alternatively the polypeptide may be produced as a fusion protein linked to the modified clostridial binding fragment by recombinant technology using the linker peptides as described above. In such an example, the construct would contain the following components:—
a polypeptide therapeutic substance;
a linker peptide; and
a modified clostridial heavy chain

An example of a polypeptide therapeutic payload is superoxide dismutase.

In yet another embodiment of the invention, the modified clostridial heavy chain is linked directly or indirectly to DNA such that the construct is capable of delivering the DNA to neuronal cells, e.g. via the receptor for tetanus toxin. Such constructs have gene therapy applications and be used to switch on, or off, selected genes with the cell. The DNA may be contained within a liposome or be condensed via a peptide or protein. The modified clostridial heavy chain may be chemically linked to the protein that effects the DNA condensation by chemical coupling agents. Alternatively, the modified clostridial heavy chain may be produced as a fusion protein, by recombinant technology, with a peptide that can effect the condensation of DNA.

In yet another embodiment of the invention, the modified clostridial heavy chain fragment may be linked to a recombinant virus such that the modified virus has an altered tropism and is capable of transducing cells via the tetanus toxin receptor. Such a construct is of use to correct genetic defects within neuronal cells by switching on, or off, selected genes. The modified clostridial heavy chain fragment may be linked directly to the surface of the virus using chemical cross-linking agents. Alternatively the modified clostridial heavy chain fragment may be linked to the recombinant virus via an antibody which specifically bind to the virus. In this instance the modified clostridial binding fragment is chemically coupled to a polyclonal or monoclonal antibody which specifically recognizes a marker on the surface of the virus. A similar modified clostridial binding fragment-antibody fusion protein could be produced by recombinant technology in which the antibody component is a recombinant single chain antibody.

In yet another embodiment of the invention, the modified clostridial heavy chain fragment is linked to a drug release system such as a microparticle constructed from a suitable polymer, e.g. poly (lactide-co-glycolide), polyhydroxylalkonate, collagen, poly(divinyl-ether-comaleic anhydride, poly (styrene-co-maleic anhydride) or other polymer useful in such microparticles. The modified clostridial heavy chain fragment may be linked to the drug release system by covalent chemical coupling, or electrostatic or hydrophobic forces. The modified clostridial heavy chain fragment may also be encapsulated within the release vehicle together with the therapeutic payload provided that a portion of the modified clostridial binding fragment is exposed at the surface. Alternatively, the modified clostridial heavy chain fragment may be linked, at either the N- or C-terminal end, to a peptide or protein to facilitate coupling of the fragment to the drug release system.

Other strategies are known by which modified heavy chain binding fragments can be linked to range of therapeutic substances using a variety of established chemical cross-linking techniques, and a variety of fusion proteins can be produced containing a modified clostridial binding fragment and another polypeptide. Using these techniques a variety of substances can be targeted to neuronal cells using the modified clostridial binding fragments. Examples of possible uses of the modified clostridial binding fragments as neuronal delivery vectors are given in more detail below in Table 1.

Constructs of the invention may be introduced into either neuronal or non-neuronal tissue using methods known in the art. By subsequent specific binding to neuronal cell tissue, the targeted construct exerts its therapeutic effects. Ideally, the construct is injected near a site requiring therapeutic intervention.

The construct of the invention may be produced as a suspension, emulsion, solution or as a freeze dried powder depending on the application and properties of the therapeutic substance. The construct of the invention may be resuspended or diluted in a variety of pharmaceutically acceptable liquids depending on the application.

“Clostridial neurotoxin” means either tetanus neurotoxin or one of the seven botulinum neurotoxins, the latter being designated as serotypes A, B C₁, D, E, F or G.

“Modified clostridial heavy chain fragment” means a polypeptide fragment which binds to neuronal cell receptors in similar manner to a corresponding heavy chain derived from botulinum or tetanus toxins but differs in its amino acid sequence and properties compared to the corresponding fragment derived from tetanus toxin.

“Bind” in relation to the botulinum and tetanus heavy chain fragments, means the specific interaction between the clostridial fragment and one or more cell surface receptors or markers which results in localization of the binding fragment on the cell surface. In the case of the clostridial neurotoxins, the property of a fragment being able to ‘bind’ like a fragment of a given serotype can be demonstrated by competition between the ligand and the native toxin for its neuronal cell receptor.

“High affinity binding specific to neuronal cell corresponding to that of a clostridial neurotoxin” refers to the ability of a ligand to bind strongly to cell surface receptors of neuronal cells that are involved in specific binding of a given neurotoxin. The capacity of a given ligand to bind strongly to these cell surface receptors may be assessed using conventional competitive binding assays. In such assays radiolabelled clostridial neurotoxin is contacted with neuronal cells in the presence of various concentrations of non-radiolabelled ligands. The ligand mixture is incubated with the cells, at low temperature (0-3° C.) to prevent ligand internalization, during which competition between the radiolabelled clostridial neurotoxin and non-labelled ligand may occur. In such assays when the unlabelled ligand used is the same as that of the labelled neurotoxin, the radiolabelled clostridial neurotoxin will be displaced from the neuronal cell receptors as the concentration of non-labelled neurotoxin is increased. The competition curve obtained in this case will therefore be representative of the behaviour of a ligand which shows “high affinity binding specificity to neuronal cells corresponding to that of a clostridial neurotoxin”, as used herein.

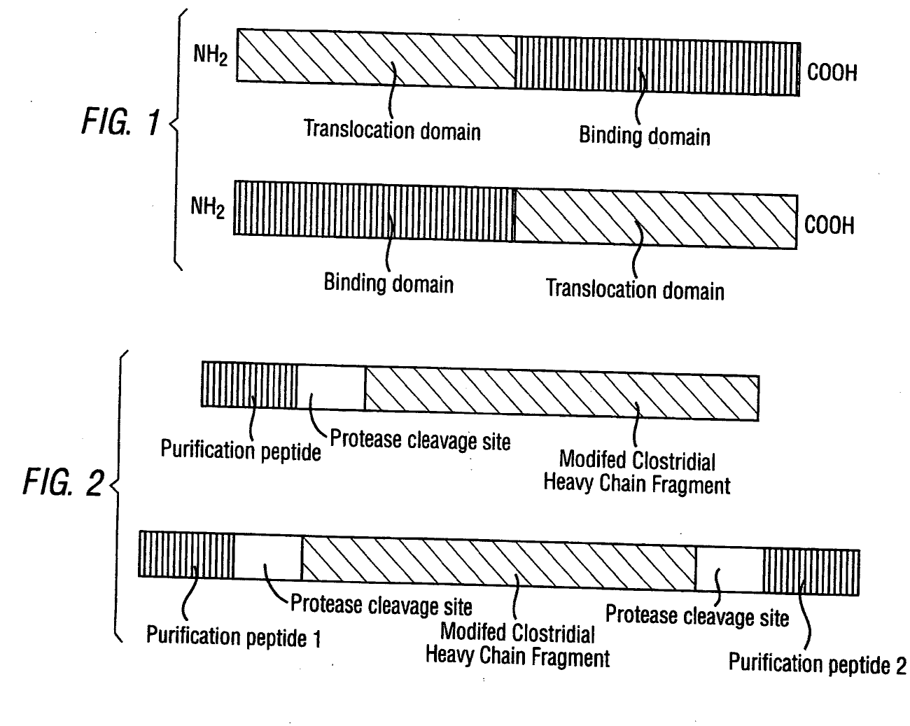

“Translocation domain” means a domain or fragment of a protein which effects transport of itself and/or other proteins and substances across a membrane or lipid bilayer. The latter membrane may be that of an endosome where translocation will occur during the process of receptor-mediated endocytosis. Translocation domains can frequently be identified by the property of being able to form measurable pores in lipid membranes at low pH (Shone et al., Eur J. Biochem. 167, 175-180). Examples of translocation domains are set out in more detail below in FIG. 1. In the application, translocation domains are frequently referred to as “H_{N} domains”.

“Translocation” in relation to translocation domain, means the internalization events which occur after binding to the cell surface. These events lead to the transport of substances into the cytosol of neuronal cells.

“Therapeutic substances” or “agents” mean any substance, agent or mixture thereof, which, if delivered by the modified clostridial binding fragment, would be beneficial to the treatment of neuronal diseases. Examples of these include drugs, growth factors, enzymes, and DNA packaged in various forms (e.g. modified viruses, cationic liposomes, and condensed DNA).

Also provided in the present invention are methods of manufacture of the polypeptides of the invention by expressing in a host cell a nucleic acid encoding the polypeptide, and the use of a polypeptide or a composition according to the invention in the treatment of a disease state associated with neuronal cells.

The invention is now illustrated in the following specific embodiments and accompanied by drawings in which:—

FIG. 1 shows modified clostridial heavy chain fragments produced by recombinant technology as a fusion proteins;

FIG. 2 shows modified clostridial heavy chain fragments produced by recombinant technology; fusion proteins may contain one or more purification peptide tags to assist in the purification of the protein; one or more protease cleavage sites may also be included to enable removal of the purification peptide tags; similar purification strategies may also be employed for modified clostridial binding fragments containing a translocation domain;

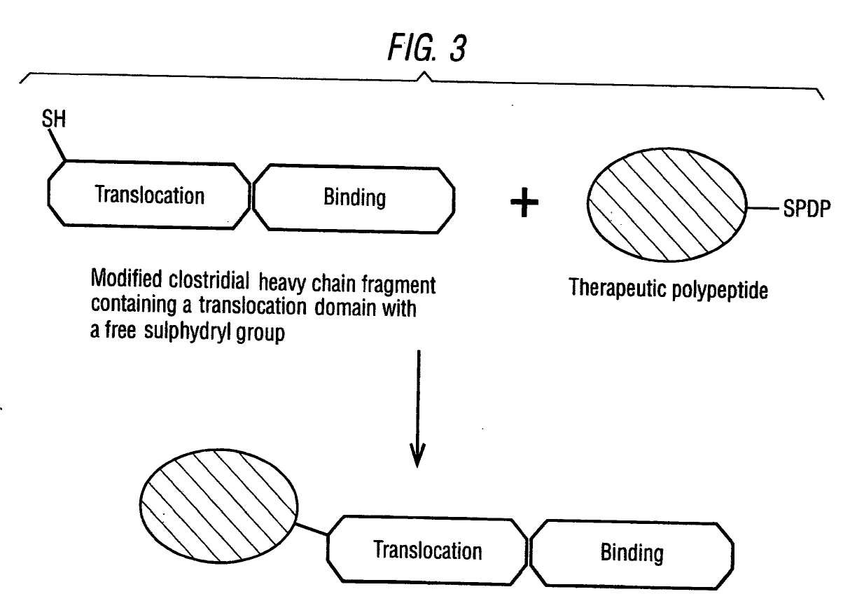

FIG. 3 shows linkage of a modified clostridial binding fragment to a therapeutic substance; the modified clostridial heavy chain contains a translocation domain which has a free thiol group (an example of translocation domain with this property is amino acid sequence 194-386 of diphtheria toxin), a free amino group on the therapeutic substance is modified with a cross-linking reagent (e.g. SPDP; Pierce & Warriner, UK Ltd.) which will subsequently allow conjugate formation using the free thiol present on the modified clostridial binding fragment;

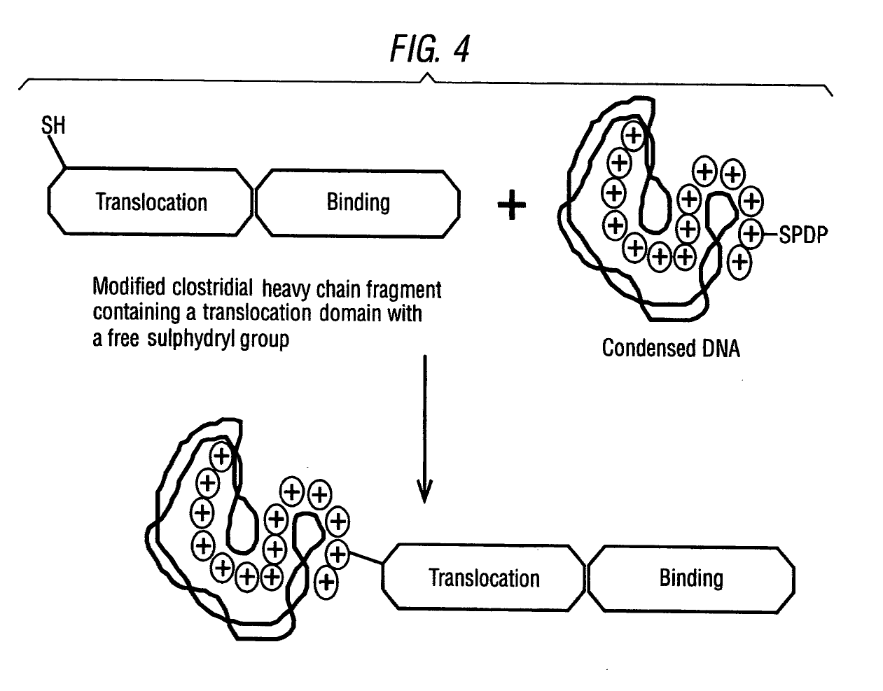

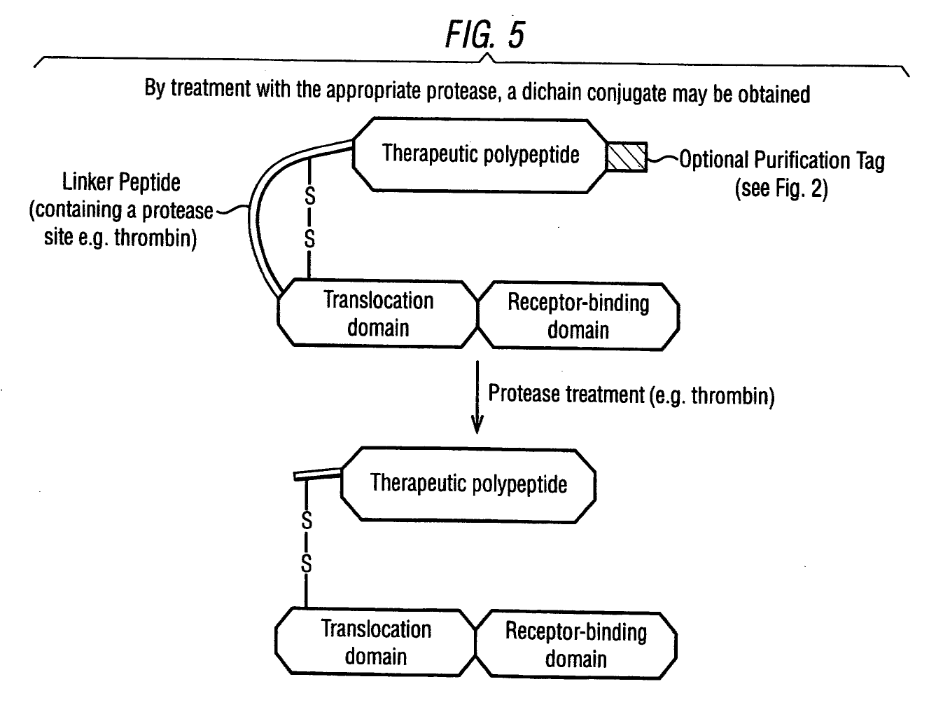

FIG. 4 shows the formation of a conjugate between a modified clostridial heavy chain fragment and an oligonucleotide as described in Example 4;

FIG. 5 shows a strategy for producing a recombinant modified clostridial heavy chain as a fusion protein with a polypeptide therapeutic substance. The latter is fused to the modified clostridial heavy chain by a linker peptide. The linker peptide contains a unique protease cleavage site (e.g. that recognized by thrombin) and a cysteine residue. Examples of linker peptides are (a) CGLVPAGSGP; and (b) CGIEGRAPGP (SEQ ID NO:18). The cysteine residue forms a disulphide bridge with an another available cysteine residue on the translocation domain of the modified heavy chain fragment. If desirable, then by treatment with thrombin, a dichain product may be produced in which the polypeptide therapeutic substance is linked to the heavy chain via a disulphide bridge;

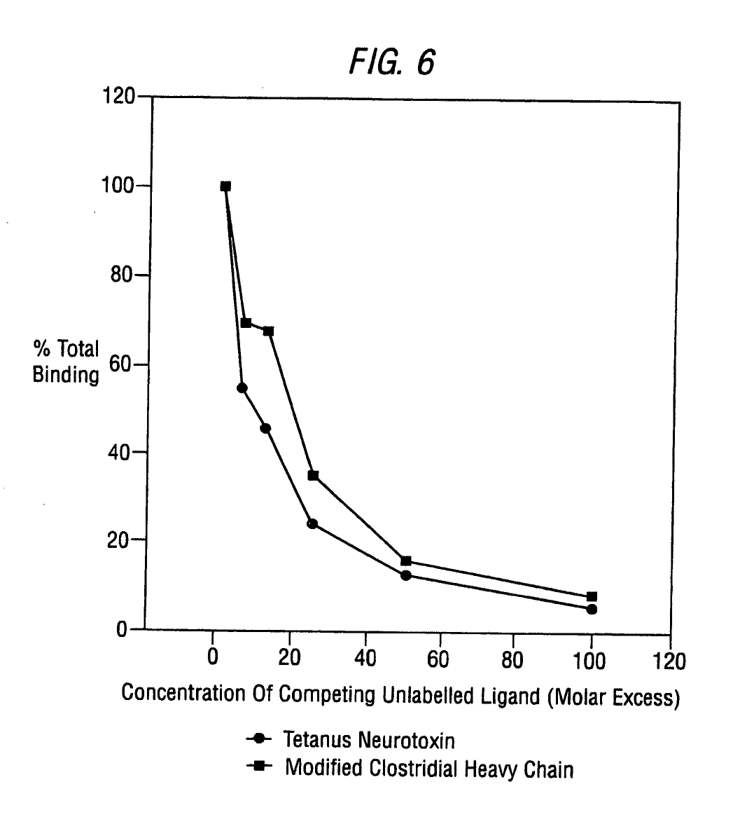

FIG. 6 shows a comparison of the binding of a modified heavy chain with that of the native neurotoxin to neuronal synaptic membranes, the modified heavy chain displaying the binding characteristics of tetanus neurotoxin as assessed by the method described in Example 7;

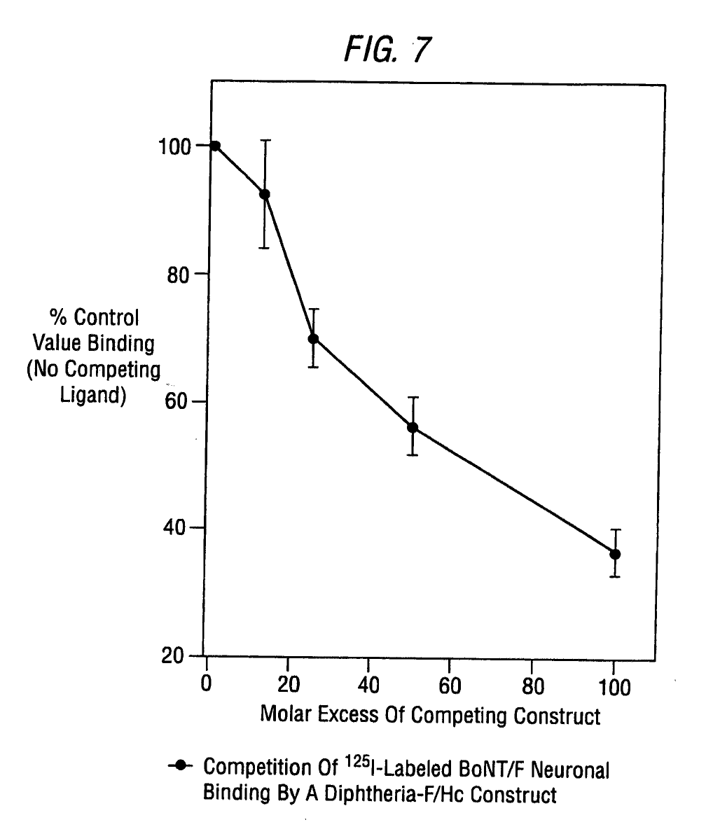

FIG. 7 shows the binding to neuronal membranes of a modified clostridial heavy chain based on the binding domain of botulinum type F neurotoxin; in this example, modified heavy chain contained the translocation (H_{N}) domain of diphtheria toxin and the binding (H_{C}) domain of type F neurotoxin; and

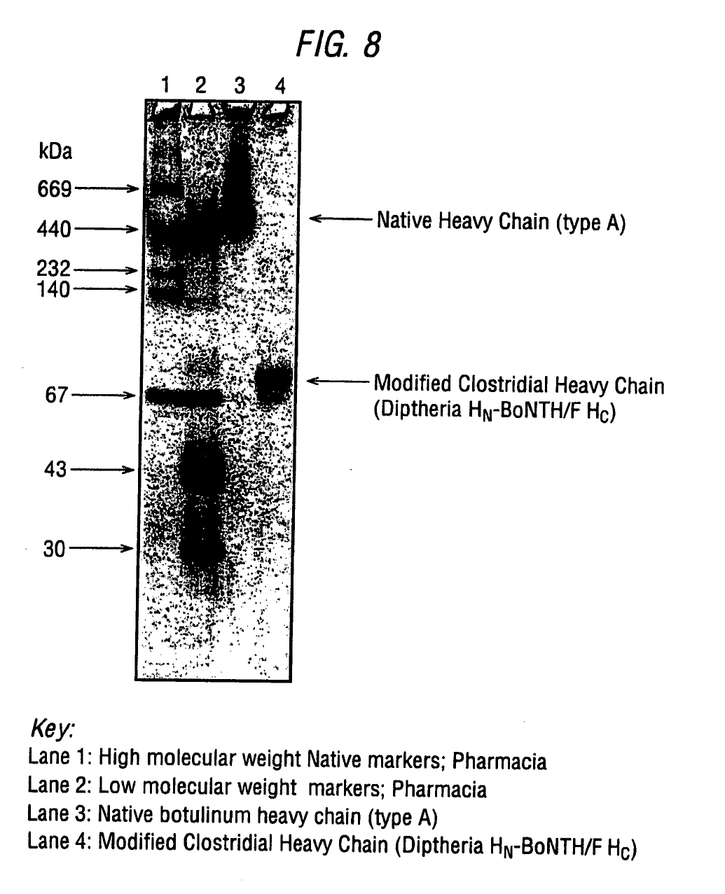

FIG. 8 shows a comparison of the molecular sizes, under non-denaturing conditions, of a modified clostridial heavy chain compared to a native heavy chain; the modified clostridial heavy chain (Diphtheria H_{N} BoNT/F H_{C}) runs as a monomer of approximately 70 kDa while a native heavy chain (from BoNT/A) runs as an aggregate of >500 kDa.

In more detail, FIG. 1 shows examples of embodiments of the invention incorporating modified clostridial heavy chain fragments.

The binding domain is derived from sequences of the clostridial neurotoxins:—
(a) H_{C} domains, e.g.
   BoNT/A residues 872-1296
   BoNT/B residues 859-1291
   BoNT/C residues 867-1291
   BoNT/D residues 863-1276
   BoNT/E residues 846-1252
   BoNT/F residues 865-1278
   BoNT/G residues 864-1297
   Tetanus residues 880-1315
(b) Hybrid H_{C} domains, e.g.
   hybrids of the H_{C} domain of BoNT/F and tetanus
(c) Truncated H_{C} domains

The translocation domain may be derived from a number of sources:—
(a) Bacterial toxins, e.g. diphtheria toxin fragment B (residues 194-386)
(b) Viral fusogenic peptides, e.g. from influenza virus haemagglutinin HA-2
(c) Synthetic membrane disrupting peptides (e.g. Plank et al., J. Biol. Chem., 269, 12918-12924).

FIG. 2 shows examples of Recombinant Modified Clostridial Heavy Chain Fragment Fusion Proteins Showing Positions of Purification Peptide Tags and Specific Protease Cleavage Sites (by treatment with the appropriate protease, the purification peptide tags may be removed from the modified clostridial binding fragment).

Examples of purification peptides tags are:
His6
S peptide
T7 peptide
Calmodulin binding peptide
Maltose binding protein

Examples of specific protease cleavage sites are:—
Thrombin
Enterokinase
Factor X

### EXAMPLE 1

### Preparation and Purification of a Recombinant Modified Clostridial Heavy Chain Fragments

Standard molecular biology protocols were used for all genetic manipulations (e.g. Sambrook et al., 1989, Molecular Cloning a Laboratory Manual, Second Edition, Cold Spring Harbor Laboratory Press, Cold Spring Harbor, N.Y.). An entirely synthetic gene encoding the H_{C} regions of botulinum toxin from C. botulinum type F (residues 865-1278) and tetanus toxin (residues 880-1315) were generated using Recursive PCR reactions (Prodromou & Pearl 1992, Protein Engineering, 5: 827-829) using self-priming oligonucleotides containing the desired sequence. The codon bias and GC/AT base ratio was adjusted for ease of expression in E. coli. Fragments were cloned sequentially into pLitmus 38 (New England Biolabs, Inc., Beverly, Mass.) to assemble the entire gene. Constructs for expression were sub-cloned into pMALc2 (NEB) replacing the BamH1-EcoR1 fragment. The ligation reactions were transformed into E. coli JM109 (Promega). Plasmid DNA was amplified, purified and screened for the presence of the appropriate sequence (Ausubel et al. 1989, Current Protocols in Molecular Biology, John Wiley & Sons, New York). Gene constructions confirmed as possessing the correct sequences were then transformed into the expression host E. coli BL21 (DE3) (Studier & Moffatt 1986, Journal of Molecular Biology, 189: 113-130).

Additional sequences for adding affinity purification tags and one or more specific protease site for the subsequent removal of these affinity tags were also included in the reading frame of the gene products.

The recombinant proteins expressed in pMAL were produced with amino-terminal maltose-binding protein tags allowing proteins to be purified by affinity chromatography on amylose resin. Briefly, cultures of E. coli BL21 (DE3) pMALc2-H_{C} were grown in Terrific broth-ampicillin (100 μgml⁻¹)-kanamycin (30 μgml⁻¹) to an OD₆₀₀ nm of 2.5-3.8, and protein expression was induced by the addition of 1 mM IPTG for approximately 2 h. Cells were lysed by freeze/thaw followed by sonication, lysates cleared by centrifugation and supernatants loaded onto an amylose resin column and eluted with maltose. All buffers used were as specified by the manufacturer. Thrombin or factor Xa protease sites were included within the protein for subsequent removal of these purification tags.

Other coding sequences which enable expression of the desired protein would also be acceptable. Other tags or linking sites may also be incorporated into the sequence. Examples of some of these options are summarized in FIG. 2.

### EXAMPLE 2

### Production of a Modified Clostridial Heavy Chain Fragments

Using the techniques described in Example 1, modified clostridial heavy chain fragments was constructed by fusing domains of the H_{C} fragments of either botulinum type F or tetanus neurotoxins with the translocation domain of diphtheria toxin. The amino acid sequences of examples are shown in SEQ ID NO:s 8-17, which also gives examples of modified tetanus heavy chains in which the H_{C} fragment is a hybrid of tetanus and botulinum type F neurotoxin.

### EXAMPLE 3

### Coupling of a Modified Clostridial Heavy Chain Fragment to a Protein or an Enzyme

The polypeptide, protein or enzyme to be linked to the modified clostridial heavy chain fragment is first derivatized with a suitable cross-linking agent. Mn-Superoxide dismutase (SOD) was modified by treatment with a 15 molar excess of SPDP (Pierce) in 0.05M Hepes buffer pH 7.0 containing 0.15M NaCl for 60 min at 25° C. The excess SPDP was removed by dialysis against the same buffer At 4° C. for 16 h. The substituted SOD was then mixed in a 1:5 molar ration with modified clostridial heavy chain fragment fused to a translocation domain derived from diphtheria toxin (see FIG. 3) and incubated at 25° C. for 16 h. After incubation the SOD-modified clostridial binding fragment conjugate was purified by gel filtration chromatography on Sephadex G200.

### EXAMPLE 4

### Coupling of Modified Clostridial Heavy Chain Fragment to Condensed DNA

Poly-L-lysine (M, 1000-4000) (10 mg) to be used for the condensation of DNA was dissolved in 2 ml of 20 mM Hepes buffer pH 7.4 containing 0.15M NaCl (HBS). To this solution 0.6 mg of Sulpho-LC-SPDP (Pierce and Warriner, UK Ltd.) was added and the mixture incubated for 30 min at 25° C. The activated poly-L-lysine was then dialysed against HBS at 4° C. using a dialysis tubing of 1000 molecular weight cut-off and then diluted to 1 mg/ml using HBS.

Condensation of DNA was carried out in glass tubes. Purified plasmid DNA containing a gene encoding a therapeutic protein (or a reporter gene) under the control of a suitable promoter (e.g. CMV immediate early, or a neuronal-specific promoter e.g. neuron-specific enolase promoter) was made 1 mg/ml in HBS and added to glass tubes followed by the activated poly-L-lysine as prepared above. Activated poly-L-lysine is added in various proportions to the DNA (see Table 2) and incubated for 90 min at 25° C.

TABLE 2
Condensation of DNA with activated poly-L-lysine.

| Sample no. | DNA (μg) | Activated Poly-L-lysine | HBS |
|---|---|---|---|
| 1 | 750 | 250 | 1500 |
| 2 | 1500 | 500 | 500 |
| 3 | 500 | 250 | 1750 |
| 4 | 1000 | 500 | 1000 |

After incubation the size of the condensed DNA particles was assessed using a Brookhaven BI90 particle sizer. The incubation conditions giving the highest proportion of condensed DNA particle of less than 100 nM in diameter was used to produce DNA-modified clostridial binding fragment conjugates. Modified clostridial heavy chain was dialysed against HBS.

The dialysed fragments (100 μg) was then added to 1 ml of condensed DNA and incubated for 18 h at 25° C. to from the modified clostridial binding protein-condensed DNA construct (see FIG. 4).

### EXAMPLE 5

### Delivery of DNA to a Neuronal Cells Via the Modified Clostridial Heavy Chain Fragment Receptor

Modified clostridial heavy chain-condensed DNA construct described in Example 4 was diluted with 2 ml MEM serum free medium. Growth media from NG108 grown in 12 well dished was removed and 1 ml of the diluted construct added and incubated for 2 h at 37° C. in the presence of 5% CO₂. Growth media (1 ml) was then added to each well and the incubation continued under the same conditions for 24-48 h. After this period the cell were examined.

In experiments were the condensed DNA contained a reporter gene encoding Green Fluorescent Protein, several of the cells showed visible expression of the reporter protein illustrating successful delivery of the DNA into the neuronal cell. Various control experiments were conducted to confirm the observed transfection in NG108 cells was receptor mediated:—

Transfection of NG108 cells was found to be dependent on the presence of modified clostridial heavy chain fragment within conjugates (no transfection was observed with condensed particles DNA alone)

No transfection was observed in non-neuronal cells (Vero cells) using the heavy chain-DNA conjugates.

### EXAMPLE 6

### Preparation of Conjugates of Modified Clostridial Heavy Chain Fragment and Microparticles Consisting of Poly (Lactide-Co-Glycolide)

398 mg of poly (lactide co-glycolide) low internal viscosity (3000 MW) (Beohringer Mannheim) was dissolved in 4 ml dichloromethane. This was homogenised at 2000 rpm for 150 seconds with 1 ml of buffer solution containing the therapeutic substance, such as an enzymes and/or drugs. In the case of Mn superoxide dismutase, 10 mg of the enzyme was dissolved in 10 mM Hepes buffer pH 8.0 containing 100 mM NaCl. The mixture was then added to 50 ml of 8% poly vinyl alcohol and emulsified at 2000 rpm for a further 150 seconds. The emulsion was poured into 300 ml of ultrapure distilled water at 37° C. and stirred for 30 min at 37° C. The microparticles were collected by centrifugation at 10000×g for 25 min at 20° C. and then resuspended in 300 ml water and centrifuged as above. This washing procedure was the repeated a further 4 times. After the final centrifugation the water supernatant fluid was removed and the microparticles freeze dried.

2 mg of poly (lactide-co-glycolide) microparticles were re-suspended in 1 ml of activation buffer (01.M MES buffer, pH 6.0 containing 0.5M NaCl). Solid 1-Ethyl-3-[3-dimethylaminopropyl]carbodiimide (EDC) and N-hydroxysulphosuccinimide (sulphoNHS) were added to 2 mM and 5 mM respectively and the mixture incubated for 15 min at 25° C. The microparticles were washed by centrifugation for 1 min at 10000×g and resuspension in 1 ml of activation buffer. The wash step was repeated 4 times and then the microparticles resuspended in 1 ml of activation buffer containing 33 μM of a modified clostridial heavy chain fragment and incubated for 2 h at 25° C. The reaction was then quenched with 10 mM hydroxylamine. After 20 min at 25° C. the microparticles were washed in a suitable buffer by centrifugation as described above.

### EXAMPLE 7

### Demonstration of the High Affinity Binding to Neuronal Cell Tissue Displayed by Modified Heavy Chain Fragments

Clostridial neurotoxins may be labelled with 125-iodine using chloramine-T and its binding to various cells assessed by standard methods such as described in Evans et al. 1986, Eur J. Biochem., 154, 409 or Wadsworth et al., 1990, Biochem. J. 268, 123). In these experiments the ability of modified clostridial heavy chain constructs to compete with native clostridial neurotoxins for receptors present on neuronal cells or brain synaptosomes was assessed. All binding experiments were carried out in binding buffers. For the botulinum neurotoxins this buffer consisted of: 50 mM hepes pH 7.0, 30 mM NaCl, 0.25% sucrose, 0.25% bovine serum albumin. For tetanus toxin, the binding buffer was: 0.05M MES buffer pH 6.0 containing 0.6% bovine serum albumin. In a typical binding experiment the radiolabelled clostridial neurotoxin was held at a fixed concentration of between 1-10 nM. Reaction mixtures were prepared by mixing the radiolabelled toxin with various concentrations of unlabelled neurotoxin or modified clostridial heavy chain construct. The reaction mixture were then added to neuronal cells or rat brain synaptosomes and then incubated at 0-3° C. for 2 hr. After this period the neuronal cells of synaptosomes were washed twice with binding ice-cold binding buffer and the amount of labelled clostridial neurotoxin bound to cells or synaptosomes was assessed by γ-counting.

In an experiment using a modified clostridial heavy construct which consisted of a binding domain derived from tetanus toxin and a translocation domain from diphtheria toxin, the construct was found to compete with ¹²⁵I-labelled tetanus neurotoxin for neuronal cell receptors in a similar manner to unlabelled native tetanus neurotoxin (see FIG. 6). These data showed that the construct had retained binding properties of the native neurotoxin.

In a further experiment using Diphtheria H_{N} BoNT/F H_{C} as the modified clostridial heavy chain, the construct was found to compete with ¹²⁵I-labelled BoNT/F for receptors on neuronal synaptic membranes (FIG. 7). These data indicate that the modified clostridial heavy chain retains the neuronal receptor-binding properties of BoNT/F.

### EXAMPLE 8

### Non-Denaturing Gel Electrophoresis to Compare the Sizes of a Native Botulinum toxin heavy chain (type A) with that of a modified clostridia, Heavy Chain (Recombinant Diphtheria H_{N}-BoNT/H_{C})

Botulinum type A heavy chain was purified as described previously (Shone et al., 1985 Eur J. Biochemistry 151, 75-82) and recombinant Diphtheria H_{N}-BoNT/F H_{C} purified as described in Examples 1 and 2. The modified clostridial heavy chain was purifies as a Maltose Binding Protein fusion with then the fusion protein removed by treatment with Factor Xa. Samples of type A heavy chain (20 μg) and Diphtheria H_{N}-BoNT/F H_{C} (10 μg) were loaded on a 4-20% Tris-glycine polyacrylamide gel in Tris-glycine buffer. Samples were electrophoresed to equilibrium (Novex gel system; 43 volts 16 hours) and the gel stained with Coomassie blue. The results are shown in FIG. 8. The major band for Diphtheria H_{N}-BoNT/F/H_{C} appears to migrate very close to its predicted molecular weight of approx 70 kDa. In contrast, the native type A heavy chain appears as a diffuse band at approximately 500 kDa, compared to an estimated molecular weight of 100 kDa, which suggesting the formation of large protein aggregates.

### EXAMPLE 9

### Recombinant Modified Heavy Chain-Superoxide Dismutase Conjugates

Recombinant modified heavy chain-superoxide dismutase conjugates were prepared comprising a combination of the following elements:—
a bacterial superoxide dismutase, from Bacillus stearothermophllus;
a linker region which allows the formation of a disulphide bond between the superoxide dismutase and the translocation domain and which also contains a unique protease cleavage site for cleavage by factor Xa or thrombin to allow the formation of a dichain molecule;
a translocation domain from diphtheria toxin or a endosomolytic (fusogenic) peptide from influenza virus haemagglutinin); and
a neuronal cell-specific binding domain from tetanus or botulinum neurotoxin type F.

The sequences of these recombinant modified heavy chain-superoxide dismutase conjugates are shown in SEQ ID NO:s 3-7.

To confirm the nature of their structure, the recombinant modified clostridial heavy chain-superoxide dismutase conjugates were converted to the dichain form by treatment with a unique protease corresponding to the cleavage site sequences within the linker region. Conjugates containing the thrombin cleavage site were treated with thrombin (20 μg per mg of conjugate) for 20 h at 37° C.; conjugates containing the factor Xa cleavage site were treated with factor Xa (20 μg per mg of conjugate) for 20 min at 22° C.

On SDS-PAGE gels, under non-reducing conditions, the conjugates appeared as a band of molecular mass approx. 120 kDa. In the presence of reducing agent (dithiothreitol) two bands were observed at approx. molecular masses 70 and 30 kDa corresponding to the modified clostridial heavy chain and superoxide dismutase respectively. These data illustrate that, after treatment with the unique protease, the conjugates consist of the latter two components which are linked by a disulphide bridge.

TABLE 1
Examples of Potential Therapeutic Uses of Modified Clostridial
Binding Fragments

| Therapeutic | | |
|---|---|---|
| Substance or | Site and Mechanism | Potential |
| Effector | of Action | Clinical Effects |
| (a) Enzymes:- | | |
| Superoxide | Reduce oxidative stress | Reduction of |
| dismutase | after stroke/injury of brain | neuronal damage |
| | or spinal cord | after ischemia/ |
| | | reperfusion |
| Glutamine | Reduce damage by excess | Reduction of |
| synthetase | glutamate after stroke/injury | neuronal damage |
| | of the brain or spinal cord | after ischemia/ |
| | | reperfusion |
| (b) Antibodies:- | | |
| Anti-tetanus toxin | Neutralize the action of | Reverse the effects |
| | tetanus toxin at the spinal | of intoxication |
| | cord | by tetanus toxin |
| Anti SNARE | Modulate neurotransmitter | Hyper secretory |
| protein | release | disorders |
| (e.g. SNAP-25, | | |
| VAMPs | | |
| Syntaxins) | | |
| © Viruses/DNA | | |
| Viral gene | Replacement of defective | Treatment of |
| therapy vectors | genes within the CNS | neurodegenerative |
| (e.g. adenovirus, | | diseases (Parkinson's' |
| herpes simplex, | | Alzheimer’s ALS etc.) |
| etc.) | | and other neuronal |
| | | diseases |
| Non-viral vectors | Replacement of defective | Treatment of |
| for gene therapy | genes within the CNS | neurodegenerative |
| (e.g. liposomes) | | diseases and other |
| | | neuronal diseases |
| (d) Growth | | |
| factors | | |
| e.g. BDNF, | Deliver growth factors to | Treatment of |
| CTNF, NGF | the brain and spinal cord | neurodegenerative |
| | | diseases, promotion |
| | | of neuronal growth |
| | | after damage. |
| (e) Anti-viral | Deliver anti-viral agents | Treatment of latent |
| agents | to the brain or spinal cord | viral infections |
| | neurons | within neuronal |
| | | cells, e.g. HIV, herpes |
| | | simplex infections |
| (f) Anti-cancer | Deliver cytotoxic agents | Treatment of neuronal |
| agents | to neoplastic cells of the CNS | neoplasia |

### Sequences of Modified Clostridial Heavy Chain—Superoxide Dismutase Conjugates

Construct containing:
MnSOD from B. stearothermophilus
a linker that can be cleaved by thrombin
a translocation domain derived from diphtheria
toxin
a binding domain from tetanus toxin
SEQ ID NO: 3
MPFELPALPYPYDALEPHIDKETMNIHHTKHHNTYVTNLNAALEGHPDL
QNKSLEELLSNLEALPESIRTAVRNNGGGHANHSLFWTILSPNGGGEPT
GELADAINKKFGSFTAFKDEFSKAAAGRFGSGWAWLVVNNGELEITSTP
NQDSPIMEGKTPILGLDVWEHAYYLKYQNRRPEYIAAFWNVVNWDEVAK
RYSEAKPKSGSCGLVPRGSGPGSSVGSSLSCINLDWDVIRDKTKTKIES
LKEHGPIKNKMSESPNKTVSEEKAKQYLEEFHQTALEHPELSELKTVTG
TNPVFAGANYAAWAVNVAQVIDSETADNLEKTTAALSILPGIGSVMGIA
DGAVHHNTEEIVAQSIALSSLMVAQAIPLVGELVDIGFAAYNFVESIIN
LFQVVHNSYNRSAYSPGHKTQPFLHDGYAVSWNTVRSKNLDCWVDNEED
IDVILKKSTILNLDINNDIISDISGFNSSVITYPDAQLVPGINGKAIHL
VNNESSEVIVHKAMDIEYNDMFNNFTVSFWLRVPKVSASHLEQYGTNEY
SIISSMKKHSLSIGSGWSVSLKGNNLIWTLKDSAGEVRQITFRDLPDKF
NAYLANKWVFITITNDRLSSANLYINGVLMGSAEITGLGAIREDNNITL
KLDRCNNNNQYVSIDKFRIFCKALNPKEIEKLYTSYLSITFLRDFWGNP
LRYDTEYYLIPVASSSKDVQLKNITDYMYLTNAPSYTNGKLNIYYRRLY
NGLKFIIKRYTPNNEIDSFVKSGDFIKLYVSYNNNEHIVGYPKDGNAFN
NLDRILRVGYNAPGIPLYKKMEAVKLRDLKTYSVQLKLYDDKNASLGLV
GTHNGQIGNDPNRDILIASNWYFNHLKDKILGCDWYFVPTDEGWTNDLQ
Construct containing:
MnSOD from B. stearothermophilus
a linker that can be cleaved by factor Xa
a translocation domain derived from diphtheria
toxin
a binding domain from botulinum type F toxin
SEQ ID NO: 4
MPFELPALPYPYDALEPHIDKETMNIHHTKHHNTYVTNLNAALEGHPDL
QNKSLEELLSNLEALPESIRTAVRNNGGGHANHSLFWTILSPNGGGEPT
GELADAINKKFGSFTAFKDEFSKAAAGRFGSGWAWLVVNNGELEITSTP
NQDSPIMEGKTPILGLDVWEHAYYLKYQNRRPEYIAAFWNVVNWDEVAK
RYSEAKPKSGSCGIEGRAPGPGSSVGSSLSCINLDWDVIRDKTKTKIES
LKEHGPIKNKMSESPNKTVSEEKAKQYLEEFHQTALEHPELSELKTVTG
TNPVFAGANYAAWAVNVAQVIDSETADNLEKTTAALSILPGIGSVMGIA
DGAVHHNTEEIVAQSIALSSLMVAQAIPLVGELVDIGFAAYNFVESIIN
LFQVVHNSYNRSAYSPGHKTQPFLHDGYAVSWNTVRSTMSYTNDKILIL
YFNKLYKKIKDNSILDMRYENNKFIDISGYGSNISINGDVYIYSTNRNQ
FGIYSSKPSEVNIAQNNDIIYNGRYQNFSISFWVRIPKYFNKVNLNNEY
TIIDCIRNNNSGWKISLNYNKIIWTLQDTAGNNQKLVFNYTQMISISDY
INKWIFVTITNNRLGNSRIYINGNLIDEKSISNLGDIHVSDNILFKIVG
CNDTRYVGIRYFKVFDTELGKTEIETLYSDEPDPSILKDFWGNYLLYNK
RYYLLNLLRTDKSITQNSNFLNINQQRGVYQKPNIFSNTRLYTGVEVII
RKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASS
WYYNNIRKNTSSNGCFWSFISKEHGWQEN
Construct containing:
a mitochondrial leader sequence from human MnSOD
MnSOD from B. stearothermophilus
a linker that can be cleaved by factor Xa
a translocation domain derived from diphtheria
toxin
a binding domain from tetanus toxin
SEQ ID NO: 5
MLSRAVCGTSRQLAPALGYLGSRQKHSRGSPALPYPYDALEPHIDKETM
NIHHTKHHNTYVTNLNAALEGHPDLQNKSLEELLSNLEALPESIRTAVR
NNGGGHANHSLFWTILSPNGGGEPTGELADAINKKFGSFTAFKDEFSKA
AAGRFGSGWAWLVVNNGELEITSTPNQDSPIMEGKTPILGLDVWEHAYY
LKYQNRRPEYIAAFWNVVNWDEVAKRYSEAKPKSGSCGIEGRAPGPGSS
VGSSLSCINLDWDVIRDKTKTKIESLKEHGPIKNKMSESPNKTVSEEKA
KQYLEEFHQTALEHPELSELKTVTGTNPVFAGANYAAWAVNVAQVIDSE
TADNLEKTTAALSILPGIGSVMGIADGAVHHNTEEIVAQSIALSSLMVA
QAIPLVGELVDIGFAAYNFVESIINLFQVVHNSYNRSAYSPGHKTQPFL
HDGYAVSWNTVRSKNLDCWVDNEEDIDVILKKSTILNLDINNDIISDIS
GFNSSVITYPDAQLVPGINGKAIHLVNNESSEVIVHKAMDIEYNDMFNN
FTVSFWLRVPKVSASHLEQYGTNEYSIISSMKKHSLSIGSGWSVSLKGN
NLIWTLKDSAGEVRQITFRDLPDKFNAYLANKWVFITITNDRLSSANLY
INGVLMGSAEITGLGAIREDNNITLKLDRCNNNNQYVSIDKFRIFCKAL
NPKEIEKLYTSYLSITFLRDFWGNPLRYDTEYYLIPVASSSKDVQLKNI
TDYMYLTNAPSYTNGKLNIYYRRLYNGLKFIIKRYTPNNEIDSFVKSGD
FIKLYVSYNNNEHIVGYPKDGNAFNNLDRILRVGYNAPGIPLYKKMEAV
KLRDLKTYSVQLKLYDDKNASLGLVGTHNGQIGNDPNRDILIASNWYFN
HLKDKILGCDWYFVPTDEGWTNDLQ
Construct containing:
a mitochondrial leader sequence from human MnSOD
MnSOD from B. stearothermophilus
a linker that can be cleaved by thrombin
a translocation domain derived from diphtheria
toxin
a binding domain from botulinum type F toxin
SEQ ID NO: 6
MLSRAVCGTSRQLAPALGYLGSRQKHSRGSPALPYPYDALEPHIDKETM
NIHHTKHHNTYVTNLNAALEGHPDLQNKSLEELLSNLEALPESIRTAVR
NNGGGHANHSLFWTILSPNGGGEPTGELADAINKKFGSFTAFKDEFSKA
AAGRFGSGWAWLVVNNGELEITSTPNQDSPIMEGKTPILGLDVWEHAYY
LKYQNRRPEYIAAFWNVVNWDEVAKRYSEAKPKSGSCGLVPRGSGPGSS
VGSSLSCINLDWDVIRDKTKTKIESLKEHGPIKNKMSESPNKTVSEEKA
KQYLEEFHQTALEHPELSELKTVTGTNPVFAGANYAAWAVNVAQVIDSE
TADNLEKTTAALSILPGIGSVMGIADGAVHHNTEEIVAQSIALSSLMVA
QAIPLVGELVDIGFAAYNFVESIINLFQVVHNSYNRSAYSPGHKTQPFL
HDGYAVSWNTVRSTMSYTNDKILILYFNKLYKKIKDNSILDMRYENNKF
IDISGYGSNISINGDVYIYSTNRNQFGIYSSKPSEVNIAQNNDIIYNGR
YQNFSISFWVRIPKYFNKVNLNNEYTIIDCIRNNNSGWKISLNYNKIIW
TLQDTAGNNQKLVFNYTQMISISDYINKWIFVTITNNRLGNSRIYINGN
LIDEKSISNLGDIHVSDNILFKIVGCNDTRYVGIRYFKVFDTELGKTEI
ETLYSDEPDPSILKDFWGNYLLYNKRYYLLNLLRTDKSITQNSNFLNIN
QQRGVYQKPNIFSNTRLYTGVEVIIRKNGSTDISNTDNFVRKNDLAYIN
VVDRDVEYRLYADISIAKPEKIIKLIRTSNSNNSLGQIIVMDSIGNNCT
MNFQNNNGGNIGLLGFHSNNLVASSWYYNNIRKNTSSNGCFWSFISKEH
GWQEN
Construct containing:
MnSOD from B. stearothermophilus
a linker that can be cleaved by factor Xa
a translocation peptide from influenza virus
a binding domain from botulinum type F toxin
SEQ ID NO: 7
MPFELPALPYPYDALEPHIDKETMNIHHTKHHNTYVTNLNAALEGHPDL
QNKSLEELLSNLEALPESIRTAVRNNGGGHANHSLFWTILSPNGGGEPT
GELADAINKKFGSFTAFKDEFSKAAAGRFGSGWAWLVVNNGELEITSTP
NQDSPIMEGKTPILGLDVWEHAYYLKYQNRRPEYIAAFWNVVNWDEVAK
RYSEAKPKSGSCGIEGRAPGPGSSVGSSLSCINGLFGAIAGFIENGWEG
MIDGWYGTMSYTNDKILILYFNKLYKKIKDNSILDMRYENNKFIDISGY
GSNISINGDVYIYSTNRNQFGIYSSKPSEVNIAQNNDIIYNGRYQNFSI
SFWVRIPKYFNKVNLNNEYTIIDCIRNNNSGWKISLNYNKIIWTLQDTA
GNNQKLVFNYTQMISISDYINKWIFVTITNNRLGNSRIYINGNLIDEKS
ISNLGDIHVSDNILFKIVGCNDTRYVGIRYFKVFDTELGKTEIETLYSD
EPDPSILKDFWGNYLLYNKRYYLLNLLRTDKSITQNSNFLNINQQRGVY
QKPNIFSNTRLYTGVEVIIRKNGSTDISNTDNFVRKNDLAYINVVDRDV
EYRLYADISIAKPEKIIKLIRTSNSNNSLGQIIVMDSIGNNCTMNFQNN
NGGNIGLLGFHSNNLVASSWYYNNIRKNTSSNGCFWSFISKEHGWQEN

Protein Sequence for Diphtheria Toxin Translocation Domain with BoNT/F-H_{C}

SEQ ID NO: 8
GSSVGSSLSCINLDWDVIRDKTKTKIESLKEHGPIKNKIMSESPNKTVS
EEKAKQYLEEFHQTALEHPELSELKTVTGTNPVFAGANYAAWAVNVAQV
IDSETADNLEKTTAALSILPGIGSVMGIADGAVHHNTEEIVAQSIALSS
LMVAQAIPLVGELVDIGFAAYNFVESIINLFQVVHNSYNRPAYSPGHKT
QPFLHDGYAVSWNTVRSTMSYTNDKILILYFNKLYKKIKDNSILDMRYE
NNKFIDISGYGSNISINGDVYIYSTNRNQFGIYSSKPSEVNIAQNNDII
YNGRYQNFSISFWVRIPKYFNKVNLNNEYTIIDCIRNNNSGWKISLNYN
KIIWTLQDTAGNNQKLVFNYTQMISISDYINKWIFVTITNNRLGNSRIY
INGNLIDEKSISNLGDIHVSDNILFKIVGCNDTRYVGIRYFKVFDTELG
KTEIETLYSDEPDPSILKDFWGNYLLYNKRYYLLNLLRTDKSITQNSNF
LNINQQRGVYQKPNIFSNTRLYTGVEVIIRKNGSTDISNTDNFVRKNDL
AYINVVDRDVEYRLYADISIAKPEKIIKLIRTSNSNNSLGQIIVMDSIG
NNCTMNFQNNNGGNIGLLGFHSNNLVASSWYYNNIRKNTSSNGCFWSFI
SKEHGWQEN

Protein Sequence for Diphtheria Toxin Translocation Domain with TeNt-H_{C}

SEQ ID NO: 9
GSSVGSSLSCINLDWDVIRDKTKTKIESLKEHGPIKNKMSESPNKTVSE
EKAKQYLEEFHQTALEHPELSELKTVTGTNPVFAGANYAAWAVNVAQVI
DSETADNLEKTTAALSILPGIGSVMGIADGAVHHNTEEIVAQSIALSSL
MVAQAIPLVGELVDIGFAAYNFVESIINLFQVVHNSYNRPAYSPGHKTQ
PFLHDGYAVSWNTVRSKNLDCWVDNEEDIDVILKKSTILNLDINNDIIS
DISGFNSSVITYPDAQLVPGINGKAIHLVNNESSEVIVHKAMDIEYNDM
FNNFTVSFWLRVPKVSASHLEQYGTNEYSIISSMKKHSLSIGSGWSVSL
KGNNLIWTLKDSAGEVRQITFRDLPDKFNAYLANKWVFITITNDRLSSA
NLYINGVLMGSAEITGLGAIREDNNITLKLDRCNNNNQYVSIDKFRIFC
KALNPKEIEKLYTSYLSITFLRDFWGNPLRYDTEYYLIPVASSSKDVQL
KNITDYMYLTNAPSYTNGKLNIYYRRLYNGLKFIIKRYTPNNEIDSFVK
SGDFIKLYVSYNNNEHIVGYPKDGNAFNNLDRILRVGYNAPGIPLYKKM
EAVKLRDLKTYSVQLKLYDDKNASLGLVGTHNGQIGNDPNRDILIASNW
YFNHLKDKILGCDWYFVPTDEGWTNDLQ

### Protein Sequence for Diphtheria Toxin Translocation Domain TeNT-H_{C} Domain II

SEQ ID NO: 10
GSSVGSSLSCINLDWDVIRDKTKTKIESLKEHGPIKNKMSESPNKTVSE
EKAKQYLEEFHQTALEHPELSELKTVTGTNPVFAGANYAAWAVNVAQVI
DSETADNLEKTTAALSILPGIGSVMGIADGAVHHNTEEIVAQSIALSSL
MVAQAIPLVGELVDIGFAAYNFVESIINLFQVVHNSYNRPAYSPGHKTQ
PFLHDGYAVSWNTVRSVYVSIDKFRIFCKALNPKEIEKLYTSYLSITFL
RDFWGNPLRYDTEYYLIPVASSSKDVQLKNITDYMYLTNAPSYTNGKLN
IYYRRLYNGLKFIIKRYTPNNEIDSFVKSGDFIKLYVSYNNNEHIVGYP
KDGNAFNNLDRILRVGYNAPGIPLYKKMEAVKLRDLKTYSVQLKLYDDK
NASLGLVGTHNGQIGNDPNRDILIASNWYFNHLKDKILGCDWYFVPTDE
GWTNDLQ

Protein Sequence for Diphtheria Toxin Translocation Domain with Truncated TeNT-H_{C}

SEQ ID NO: 11
GSSVGSSLSCINLDWDVIRDKTKTKIESLKEHGPIKNKMSESPNKTVSE
EKAKQYLEEFHQTALEHPELSELKTVTGTNPVFAGANYAAWAVNVAQVI
DSETADNLEKTTAALSILPGIGSVMGIADGAVHHNTEEIVAQSIALSSL
MVAQAIPLVGELVDIGFAAYNFVESIINLFQVVHNSYNRPAYSPGHKTQ
PFLHDGYAVSWNTVRSVYNNESSEVIVHKAMDIEYNDMFNNFTVSFWLR
VPKVSASHLEQYGTNEYSIISSMKKHSLSIGSGWSVSLKGNNLIWTLKD
SAGEVRQITFRDLPDKFNAYLANKWVFITITNDRLSSANLYINGVLMGS
AEITGLGAIREDNNITLKLDRCNNNNQYVSIDKFRIFCKALNPKEIEKL
YTSYLSITFLRDFWGNPLRYDTEYYLIPVASSSKDVQLKNITDYMYLTN
APSYTNGKLNIYYRRLYNGLKFIIKRYTPNNEIDSFVKSGDFIKLYVSY
NNNEHIVGYPKDGNAFNNLDRILRVGYNAPGIPLYKKMEAVKLRDLKTY
SVQLKLYDDKNASLGLVGTHNGQIGNDPNRDILIASNWYFNHLKDKILG
CDWYFVPTDEGWTNDLQ

### Protein Sequence for Diphtheria Toxin Translocation Domain BoNT/F-H_{C} Domain I TeNT-H_{C} Domain II

SEQ ID NO: 12
GSSVGSSLSCINLDWDVIRDKTKTKIESLKEHGPIKNKMSESPNKTVSE
EKAKQYLEEFHQTALEHPELSELKTVTGTNPVFAGANYAAWAVNVAQVI
DSETADNLEKTTAALSILPGIGSVMGIADGAVHHNTEEIVAQSIALSSL
MVAQAIPLVGELVDIGFAAYNFVESIINLFQVVHNSYNRPAYSPGHKTQ
PFLHDGYAVSWNTVRSTMSYTNDKILILYFNKLYKKIKDNSILDMRYEN
NKFIDISGYGSNISINGDVYIYSTNRNQFGIYSSKPSEVNIAQNNDIIY
NGRYQNFSISFWVRIPKYFNKVNLNNEYTIIDCIRNNNSGWKISLNYNK
IIWTLQDTAGNNQKLVFNYTQMISISDYINKWIFVTITNNRLGNSRIYI
NGNLIDEKSISNLGDIHVSDNILFKIVGCNDTRYVSIDKFRIFCKALNP
KEIEKLYTSYLSITFLRDFWGNPLRYDTEYYLIPVASSSKDVQLKNITD
YMYLTNAPSYTNGKLNIYYRRLYNGLKFIIKRYTPNNEIDSFVKSGDFI
KLYVSYNNNEHIVGYPKDGNAFNNLDRILRVGYNAPGIPLYKKMEAVKL
RDLKTYSVQLKLYDDKNASLGLVGTHNGQIGNDPNRDILIASNWYFNHL
KDKILGCDWYFVPTDEGWTNDLQ

### Protein Sequence for Diphtheria Toxin Translocation Domain

SEQ ID NO: 13
SVGSSLSCINLDWDVIRDKTKTKIESLKEHGPIKNKMSESPNKTVSEEK
AKQYLEEFHQTALEHPELSELKTVTGTNPVFAGANYAAWAVNVAQVIDS
ETADNLEKTTAALSILPGIGSVMGIADGAVHHNTEEIVAQSIALSSLMV
AQAIPLVGELVDIGFAAYNFVESIINLFQVVHNSYNRPAYSPGHKTQPF
LHDGYAVSWNTVRS

Protein Sequence for Clostridium botulinum C2 Enterotoxin Translocation Domain with BoNT/F-H_{C}

(a)
SEQ ID NO: 14
LVSKFENSVKNSNKNYFTINGLMGYYFENDFFNLNIISPTLDGNLTFSK
EDINSILGNKIIKSARWIGLIKPSITGEYILSTNSPNCRVELNGEIFNL
SLNTSNTVNLIQGNVYDIRIEQLMSENQILLKNYEGIKLYWETSDIIKE
IIPSEVLLKPNYSNTNEKSKFIPNNTLFSNAKLKANANRDTDRDGIPDE
WEINGYTVMNQKAVAWDDKFAANGYKKYVSNPFKPCTANDPYTDFEKVS
GQIDPSVSMVARDPMISAYPIVGVQMERLVVSKSETITGDSTKSMSKST
SHSSTNINTVGAEVSGSLQLAGGIFPVFSMSASANYSHTWQNTSTVDDT
TGESFSQGLSINTGESAYINPNIRYYNTGTAPVYNVTPTTTIVIDKQSV
ATIKGQESLIGDYLNPGGTYPIIGEPPMALNTMDQFSSRLIPINYNQLK
SIDNGGTVMLSTSQFTGNFAKYNSNGNLVTDGNNWGPYLGTIKSTTASL
TLSFSGQTTQVAVVAPNFSDPEDKTPKLTLEQALVKAFALEKKNGKFYF
HGLEISKNEKIQVFLDSNTNNDFENQLKNTADKDIMHCIIKRNMNILVK
VITFKENISSINIINDTNFGVQSMTGLSNRSKGQDGIYRAATTAFSFKS
KELKYPEGRYRMRFVIQSYEPFTTMSYTNDKILILYFNKLYKKIKDNSI
LDMRYENNKFIDISGYGSNISINGDVYIYSTNRNQFGIYSSKPSEVNIA
QNNDIIYNGRYQNFSISFWVRIPKYFNKVNLNNEYTIIDCIRNNNSGWK
ISLNYNKIIWTLQDTAGNNQKLVFNYTQMISISDYINKWIFVTITNNRL
GNSRIYINGNLIDEKSISNLGDIHVSDNILFKIVGCNDTRYVGIRYFKV
FDTELGKTEIETLYSDEPDPSILKDFWGNYLLYNKRYYLLNLLRTDKSI
TQNSNFLNINQQRGVYQKPNIFSNTRLYTGVEVIIRKNGSTDISNTDNF
VRKNDLAYINVVDRDVEYRLYADISIAKPEKIIKLIRTSNSNNSLGQII
VMDSIGNNCTMNFQNNNGGNIGLLGFHSNNLVASSWYYNNIRKNTSSNG
CFWSFISKEHGWQEN
(b)
SEQ ID NO: 15
LVSKFENSVKNSNKNYFTINGLMGYYFENDFFNLNIISPTLDGNLTFSK
EDINSILGNKIIKSARWIGLIKPSITGEYILSTNSPNCRVELNGEIFNL
SLNTSNTVNLIQGNVYDIRIEQLMSENQLLKNYEGIKLYWETSDIIKEI
IPSEVLLKPNYSNTNEKSKFIPNNTLFSNAKLKANANRDTDRDGIPDEW
EINGYTVMNQKAVAWDDKFAANGYKKYVSNPFKPCTANDPYTDFEKVSG
QIDPSVSMVARDPMISAYPIVGVQMERLVVSKSETITGDSTKSMSKSTS
HSSTNINTVGAEVSGSLQLAGGIFPVFSMSASANYSHTWQNTSTVDDTT
GESFSQGLSINTGESAYINPNIRYYNTGTAPVYNVTPTTTIVIDKQSVA
TIKGQESLIGDYLNPGGTYPIIGEPPMALNTMDQFSSRLIPINYNQLKS
IDNGGTVMLSTSQFTGNFAKYNSNGNLVTDGNNWGPYLGTIKSTTASLT
LSFSGQTTQVAVVAPNFSDPEDKTPKLTLEQALVKAFALEKKNGKFYFH
GLEISKNEKIQVFLDSNTNNDFENQLKNTADKDIMHCIIKRNMNILVKV
ITFKENISSINTMSYTNDKILILYFNKLYKKIKDNSILDMRYENNKFID
ISGYGSNISINGDVYIYSTNRNQFGIYSSKPSEVNIAQNNDIIYNGRYQ
NFSISFWVRIPKYFNKVNLNNEYTIIDCIRNNNSGWKISLNYNKIIWTL
QDTAGNNQKLVFNYTQMISISDYINKWIFVTITNNRLGNSRIYINGNLI
DEKSISNLGDIHVSDNILFKIVGCNDTRYVGIRYFKVFDTELGKTEIET
LYSDEPDPSILKDFWGNYLLYNKRYYLLNLLRTDKSITQNSNFLNINQQ
RGVYQKPNIFSNTRLYTGVEVIIRKNGSTDISNTDNFVRKNDLAYINVV
DRDVEYRLYADISIAKPEKIIKLIRTSNSNNSLGQIIVMDSIGNNCTMN
FQNNNGGNIGLLGFHSNNLVASSWYYNNIRKNTSSNGCFWSFISKEHGW
QEN

Protein Sequence for Clostridium botulinum C2 Enterotoxin Translocation Domain with Tetanus-H_{C}

(a)
SEQ ID NO: 16
LVSKFENSVKNSNKNYFTINGLMGYYFENDFFNLNIISPTLDGNLTFSK
EDINSILGNKIIKSARWIGLIKPSITGEYILSTNSPNCRVELNGEIFNL
SLNTSNTVNLIQGNVYDIRIEQLMSENQLLKNYEGIKLYWETSDIIKEI
IPSEVLLKPNYSNTNEKSKFIPNNTLFSNAKLKANANRDTDRDGIPDEW
EINGYTVMNQKAVAWDDKFAANGYKKYVSNPFKPCTANDPYTDFEKVSG
QIDPSVSMVARDPMISAYPIVGVQMERLVVSKSETITGDSTKSMSKSTS
HSSTNINTVGAEVSGSLQLAGGIFPVFSMSASANYSHTWQNTSTVDDTT
GESFSQGLSINTGESAYINPNIRYYNTGTAPVYNVTPTTTIVIDKQSVA
TIKGQESLIGDYLNPGGTYPIIGEPPMALNTMDQFSSRLIPINYNQLKS
IDNGGTVMLSTSQFTGNFAKYNSNGNLVTDGNNWGPYLGTIKSTTASLT
LSFSGQTTQVAVVAPNFSDPEDKTPKLTLEQALVKAFALEKKNGKFYFH
GLEISKNEKIQVFLDSNTNNDFENQLKNTADKDIMHCIIKRNMNILVKV
ITFKENISSINIINDTNFGVQSMTGLSNRSKGQDGIYRAATTAFSFKSK
ELKYPEGRYRMRFVIQSYEPFTKNLDCWVDNEEDIDVILKKSTILNLDI
NNDIISDISGFNSSVITYPDAQLVPGINGKAIHLVNNESSEVIVHKAMD
IEYNDMFNNFTVSFWLRVPKVSASHLEQYGTNEYSIISSMKKHSLSIGS
GWSVSLKGNNLIWTLKDSAGEVRQITFRDLPDKFNAYLANKWVFITITN
DRLSSANLYINGVLMGSAEITGLGAIREDNNITLKLDRCNNNNQYVSID
KFRIFCKALNPKEIEKLYTSYLSITFLRDFWGNPLRYDTEYYLIPVASS
SKDVQLKNITDYMYLTNAPSYTNGKLNIYYRRLYNGLKFIIKRYTPNNE
IDSFVKSGDFIKLYVSYNNNEHIVGYPKDGNAFNNLDRILRVGYNAPGI
PLYKKMEAVKLRDLKTYSVQLKLYDDKNASLGLVGTHNGQIGNDPNRDI
LIASNWYFNHLKDKILGCDWYFVPTDEGWTNDLQ
(b)
SEQ ID NO: 17
LVSKFENSVKNSNKNYFTINGLMGYYFENDFFNLNIISPTLDGNLTFSK
EDINSILGNKIIKSARWIGLIKPSITGEYILSTNSPNCRVELNGEIFNL
SLNTSNTVNLIQGNVYDIRIEQLMSENQLLKNYEGIKLYWETSDIIKEI
IPSEVLLKPNYSNTNEKSKFIPNNTLFSNAKLKANANRDTDRDGIPDEW
EINGYTVMNQKAVAWDDKFAANGYKKYVSNPFKPCTANDPYTDFEKVSG
QIDPSVSMVARDPMISAYPIVGVQMERLVVSKSETITGDSTKSMSKSTS
HSSTNINTVGAEVSGSLQLAGGIFPVFSMSASANYSHTWQNTSTVDDTT
GESFSQGLSINTGESAYINPNIRYYNTGTAPVYNVTPTTTIVIDKQSVA
TIKGQESLIGDYLNPGGTYPIIGEPPMALNTMDQFSSRLIPINYNQLKS
IDNGGTVMLSTSQFTGNFAKYNSNGNLVTDGNNWGPYLGTIKSTTASLT
LSFSGQTTQVAVVAPNFSDPEDKTPKLTLEQALVKAFALEKKNGKFYFH
GLEISKNEKIQVFLDSNTNNDFENQLKNTADKDIMHCIIKRNMNILVKV
ITFKENISSINKNLDCWVDNEEDIDVILKKSTILNLDINNDIISDISGF
NSSVITYPDAQLVPGINGKAIHLVNNESSEVIVHKAMDIEYNDMFNNFT
VSFWLRVPKVSASHLEQYGTNEYSIISSMKKHSLSIGSGWSVSLKGNNL
IWTLKDSAGEVRQITFRDLPDKFNAYLANKWVFITITNDRLSSANLYIN
GVLMGSAEITGLGAIREDNNITLKLDRCNNNNQYVSIDKFRIFCKALNP
KEIEKLYTSYLSITFLRDFWGNPLRYDTEYYLIPVASSSKDVQLKNIDT
YMYLTNAPSYTNGKLNIYYRRLYNGLKFIIKRYTPNNEIDSFVKSGDFI
KLYVSYNNNEHIVGYPKDGNAFNNLDRILRVGYNAPGIPLYKKMEAVKL
RDLKTYSVQLKLYDDKNASLGLVGTHNGQIGNDPNRDILIASNWYFNHL
KDKILGCDWYFVPTDEGWTNDLQ

1. A composition comprising a therapeutic agent linked to a non-toxic delivery polypeptide, wherein the delivery polypeptide is for delivery of said therapeutic agent to a neuronal cell, said delivery polypeptide comprising:
(a) a binding domain that binds to the neuronal cell, and
(b) a translocation domain that translocates the therapeutic agent into the neuronal cell; and
wherein the therapeutic agent is an anti-SNARE protein antibody.

2. A composition according to claim 1, wherein the translocation domain is a non-aggregating translocation domain as measured by size in physiological buffers.

3. A composition according to claim 1, wherein the translocation domain is selected from (1) a H_{N} domain of a diphtheria toxin, (2) a fragment or derivative of (1) that substantially retains the translocating activity of the H_{N} domain of a diphtheria toxin, (3) a fusogenic peptide, (4) a membrane disrupting peptide, and (5) translocating fragments and derivatives of (3) and (4).

4. A composition according to claim 1, wherein the delivery polypeptide has the binding specificity of tetanus toxin and reduced affinity to neutralising antibodies to tetanus toxin compared with the affinity to such antibodies of native tetanus toxin heavy chain.

5. A composition according to claim 1, wherein the translocation domain is not a H_{N} domain of a clostridial toxin and is not a fragment or derivative of a H_{N} domain of a clostridial toxin.

6. A composition according to claim 1, wherein the delivery polypeptide has reduced affinity to neutralising antibodies to tetanus toxin compared with the affinity to such antibodies of native tetanus toxin heavy chain.

7. A composition according to claim 1 wherein the binding domain comprises a botulinum H_{C} domain.

8. A composition according to claim 1, wherein the binding domain comprises a tetanus H_{C} domain.

9. A composition according to claim 1, wherein the binding domain comprises a hybrid of a botulinum H_{C} domain and a tetanus H_{C} domain.

10. A composition according to claim 1, wherein said delivery polypeptide comprises a tetanus H_{C} domain and a diphtheria H_{N} domain.

11. A composition according to claim 1, wherein said delivery polypeptide comprises a botulinum H_{C} domain and diphtheria H_{N} domain.

12. A composition according to claim 1, wherein the therapeutic agent is chemically bound to said polypeptide.

13. A composition according to claim 1, wherein the therapeutic agent is linked to a translocation domain of said polypeptide.

14. A composition according to claim 1, wherein the therapeutic agent is produced as a fusion protein by recombinant technology.

15. A method of modulating neurotransmitter release from a neuronal cell, the method comprising:
contacting the cell with a composition comprising a therapeutic agent linked to a non-toxic delivery polypeptide;
wherein the therapeutic agent is an anti-SNARE protein antibody; and
wherein the delivery polypeptide comprises:
(a) a binding domain that binds to the neuronal cell, and
(b) a translocation domain that translocates the therapeutic agent into the neuronal cell.

16. The method according to claim 15, wherein the translocation domain is selected from (1) a H_{N} domain of a diphtheria toxin, (2) a fragment or derivative of an H_{N} domain of a diphtheria toxin that substantially retains the translocating activity of the H_{N} domain of a diphtheria toxin, (3) a fusogenic peptide, (4) a membrane disrupting peptide, and (5) translocating fragments and derivatives of (3) and (4).

17. The method according to claim 15, wherein the binding domain comprises a botulinum H_{C} domain, a tetanus H_{C} domain, or a hybrid of a botulinum H_{C} domain and a tetanus H_{C} domain.

18. The method according to claims 15, wherein the delivery polypeptide comprises a tetanus H_{C} domain and a diphtheria H_{N} domain.

19. The method according to claims 15, wherein the delivery polypeptide comprises a botulinum H_{C} domain and diphtheria H_{N} domain.

20. A method of treating a hyper-secretory disorder, the method comprising administering to a patient in need thereof a composition comprising a therapeutic agent linked to a non-toxic delivery polypeptide;
wherein the therapeutic agent is an anti-SNARE protein antibody; and
wherein the delivery polypeptide comprises:
(a) a binding domain that binds to the neuronal cell, and
(b) a translocation domain that translocates the therapeutic agent into the neuronal cell.
